(19) United States
(12) Patent Application Publication
(10) Pub. No.: US 2007/0277433 A1
(43) Pub. Date: Dec. 6, 2007

(54) Seedling protection device, system and method

(75) Inventor: David N. Stearns, Canby, OR (US)

Correspondence Address: ATER WYNNE LLP, 222 SW COLUMBIA, SUITE 1800, PORTLAND, OR, 97201-6618, US

(73) Assignee: IFA Nurseries, Inc.

(21) Appl. No.: 11/446,328

(22) Filed: Jun. 1, 2006

(51) Int. Cl. A01H 3/00

(52) U.S. Cl. 47/58.1 R

(57) ABSTRACT

A seedling protection device includes a flexible strip of thin flat material having opposing surfaces and opposing ends, the strip being flexible to define an interior one of the opposing surfaces and to produce an intermediate overlapping interior region thereof, the strip including an adhesive at least in the intermediate overlapping interior region, the strip having a laminar cover extending substantially over the interior one of the opposing surfaces to protect the adhesive, pre-deployment, the laminar cover being removable from the interior one of the opposing surfaces in a deployment phase of use, the strip including on at least one surface region adjacent one or more opposing ends thereof a scented compound, the strip with the laminar cover removed and when flexed, during deployment, forming a collar configured to extend around and affix to a seedling stem near an extremity thereof. A system and method for its use alone or in combination with a recently patented bud capping device are also described and illustrated.

### BACKGROUND OF THE INVENTION

This invention relates generally to the field of protecting tree seedlings from browsers. More particularly, it concerns mechanisms for extending the protection of seedlings from browsers, from an early to a later stage in the seedling's maturity.

Seedlings are typically started in nurseries where they are protected against the elements including animal browsing. However, when the seedlings are transplanted to an outdoor reforestation site or tree farm, they become vulnerable to animal browsing. Browsing damage by vertebrates can affect the growth rate and form of seedlings. If the growth rate is reduced, the time to yield or the yield itself is reduced. If browsing of the apical bud occurs then double leaders are the result, which lowers the quality and form of the final product. Control of browsing is essential to avoid partial or even complete loss of planting stock, and economic failure of a plantation. Seedlings can be protected through a range of lethal and non-lethal methods. When native animals are implicated in causing damage, non-lethal methods should be considered first. Hunting and poisoning are lethal methods of controlling browsing, but require sustained effort, can damage ecosystem balance, and can lead to secondary deaths. Trapping is a non-lethal control but requires permits and is disruptive to the animal and likely leads to animal death. Various fencing methods are effective but are costly.

Alternatively, placing protective devices, e.g. caps, on seedling buds has been found to discourage browsing, as has the use of repellents that may be sprayed on the seedlings. Previously known protective devices require the use of a staple to hold opposite edges of a piece of paper stock together, forming a cylindrical tube around the terminus of a seedling bud. Unfortunately, such crude prior art devices require a forester or nursery staff member to carry around a bulky and difficult-to-use stapler. Worse, the stapling operation is typically performed by wrapping the seedling terminus with the paper stock and then stapling the edges together. Often the staple penetrates the seedling's stem, thereby damaging the seedling. Always, the staple penetrates the paper stock, thereby diminishing its water-repellent capability. These prior art approaches did not incorporate repellent or scenting into a capping device as a form of double browse prevention.

A more recent seedling bud capping device has been developed that can be easily deployed on the terminus of a bud of a seedling due to the use of adherent on either edge (rather than a staple), and more particularly due to its predefined selective use of adherent to form an inverted conic space around the bud for receiving sunlight and for capturing the bud stem near its base. The paper stock or the adherent used in the bud capping device may be impregnated with a repellent scent to further ward away browsers to protect the seedling. Preferably, the bud capping device is made from a small, e.g. ˜5 inch, approximately square piece of water-repellent paper stock having a layer of glue adjacent at least one edge. The stock is protected on the adherent side by another layer of material to be selectively removed. The bud capping device prevents browsing of a fragile seedling while permitting the seedling to thrive. The bud capping device may also be scented, preferably with a time-release animal repellent, to further discourage browsing. Such a device is described in U.S. Pat. No. 6,843,187 B2 entitled METHOD AND DEVICE FOR CAPPING SEEDLING BUDS, which patent is subject to common ownership herewith by IFA Nurseries, Inc. of Canby, Oreg., USA. Familiarity with that patent's disclosure is assumed. The seedling protective device disclosed therein will be referred to herein as the BudCap™ seedling protective device, exclusive trademark thereto being owned and reserved world-wide by IFA Nurseries, Inc.

The bud capping device described in the above-referenced patent is especially effective with very young seedlings, but its effectiveness diminishes somewhat over time as the seedling literally outgrows it, i.e. grows through it, so that the device no longer is near the top of the maturing seedling near the site of the most vulnerable new growth.

Most BudCap™ devices to date have been sold without animal repellent, relying instead on their novel physical configuration and ease of deployment for their popularity and sales. Moreover, price point is a major issue with nurseries and foresters alike, and a cheaper unit price for viable deterrence to browsing would sell many more units. Finally, the BudCap™ product has a limited useful life and must be replaced near the terminal stem of the seedling periodically, further increasing the cost of deterrence during the vulnerable seedling growth months.

### SUMMARY OF THE INVENTION

A seedling protection device includes a flexible strip of thin flat material having opposing surfaces and opposing ends, the strip being flexible to define an interior one of the opposing surfaces and to produce an intermediate overlapping interior region thereof, the strip including an adhesive at least in the intermediate overlapping interior region, the strip having a laminar cover extending substantially over the interior one of the opposing surfaces to protect the adhesive, pre-deployment, the laminar cover being removable from the interior one of the opposing surfaces in a deployment phase of use, the strip including on at least one surface region adjacent one or more opposing ends thereof a scented compound, the strip with the laminar cover removed and when flexed, during deployment, forming a collar configured to extend around and affix to a seedling stem near an extremity thereof. A system and method for its use alone or in combination with a recently patented bud capping device are also described and illustrated.

### BRIEF DESCRIPTION OF THE DRAWINGS

FIGS. 1A and 1B are respective top and bottom isometric views of an undeployed seedling protection device in accordance with one embodiment of the invention. FIG. 1B shows the device with its protective laminar cover lifted at either end for illustration purposes only (to reveal its internal construction).

FIG. 2 is an isometric view of the invented system including the device of FIGS. 1A and 1B formed and in place on a seedling, the seedling already having a BudCap™ seedling protection device thereon.

FIG. 3 is an isometric view of removably connected plural ones of the invented protective devices in a dispensing roll.

FIG. 4 is a flowchart of the method in accordance with one embodiment of the invention.

FIG. 5 is a system block diagram showing the system in accordance with one embodiment of the invention.

### DETAILED DESCRIPTION OF THE PREFERRED EMBODIMENTS

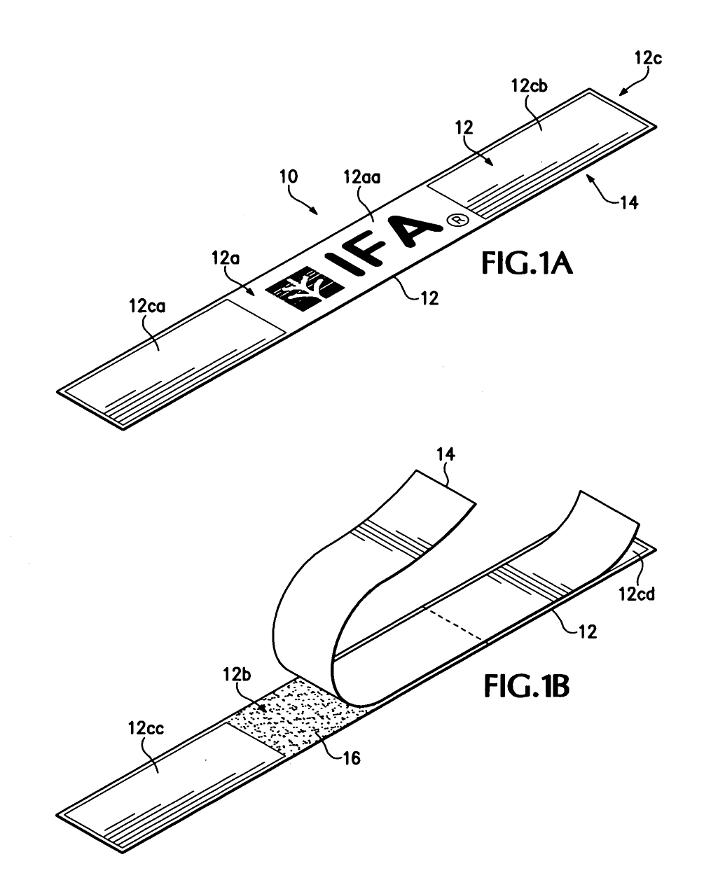

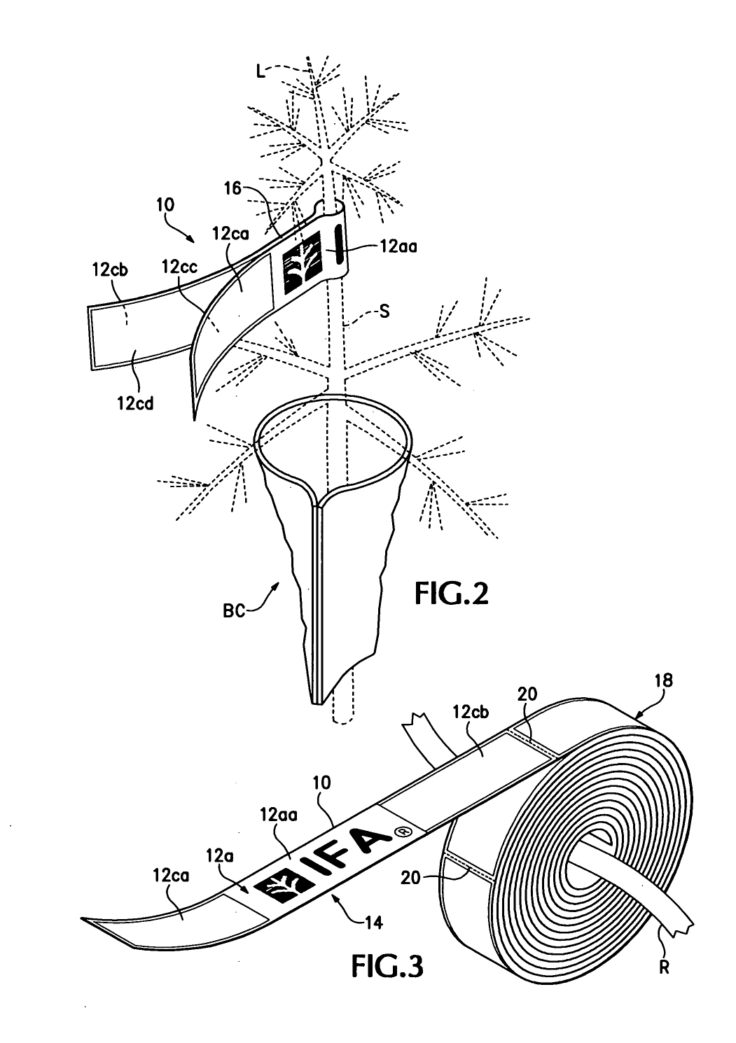

FIGS. 1A and 1B collectively illustrate in respective top and bottom isometric views an undeployed seedling protective device 10. Device 10 in its undeployed condition can be seen to be a flat thin strip of material having a substrate 12 and a removable laminar cover 14. Cover 14 adheres to an adhesive region 16 on one surface 12a of substrate 12 and is easily removed when the device 10 is deployed around a seedling, as will be seen by reference to FIG. 2. Those of skill will appreciate from FIG. 1B that, for the sake of clarity, cover 14 is shown to be slightly narrower than substrate 12, although in accordance with the embodiment described and illustrated herein, the substrate and cover are coextensive in both directions to ensure full coverage of the adhesive and to avoid difficulty in rolling or deploying the devices, as will be clear. Those of skill also will appreciate from FIG. 1B that either end of cover 14 is lifted for illustration purposes only to reveal the internal construction of device 10. Normally, it is understood that device 10 including substrate 12 and cover 14 lie flat until deployed around a seedling stem.

On the other surface 12b of substrate 12 a region 12aa generally opposite adhesive region 16 is reserved for printed matter, e.g. supplier indicia in the form, for example, of a company name, trademark, logo and/or notice (e.g. “Patent Pending”) such as the illustrated ones belonging to IFA Nurseries, Inc. Also on at least one surface 12b is at least one region 12c reserved for application of a scent, e.g. an animal repellent.

Those of skill in the art will appreciate that in accordance with one embodiment of the invention, both opposing surfaces 12a and 12b in two different terminal regions thereof are provided with scent, thereby greatly to increase the amount of animal repellent power available in device 10. Thus, in accordance with this embodiment, region 12c is configured in four separate regions, 12ca, 12cb, 12cc and 12cd, all of which are scented and half of which are scented on two different ends and another half of which are scented on two different surfaces of substrate 12. As will be seen, when device 10 having these scented regions is deployed around a terminal stem of a seedling, all four scented regions 12ca, 12cb, 12cc and 12cd are exposed to the environment, thus greatly increasing the repellent surface area and thus the browser-deterrent effect of the protective device.

Device 10 is suitably dimensioned, as will be seen, to extend around the stem of a seedling near an extremity thereof. Thus, in accordance with one embodiment of the invention, it is approximately 1″ wide and 8″ long. Its thickness depends upon the material used for the substrate and laminar cover, and in accordance with one embodiment of the invention in which the substrate and cover are made of water-resistant paper stock, its thickness is approximately 5-10 millimeters (mm). Thus, when deployed around a seedling's stem, approximately 3.75″ of either end extend from the stem and permit excellent adherence in adhesive region 16 between the inner folded surfaces. Meanwhile, approximately 2.5″ of each of the opposing ends of substrate 12 splay apart and extend radially outwardly from the adhered central region thereof fully exposing all four scented regions 12ca, 12cb, 12cc and 12cd to the environment and to a browsing animal, e.g. a deer.

Thus, the effective exposed scented repellent surface area in accordance with one embodiment of the invention is approximately 10 in², an area that can within the scope and spirit of the invention be decreased or increased to decrease or increase the deterrent effect to animal browsing. Such a large surface area for such a small strip is cleverly rendered possible by the deployment of the strip in a splayed-ends configuration that effectively doubles or quadruples the effective exposed scent surface area. Nevertheless, cost is relatively low, since the overall size in surface are of laminated paper stock of device 10 is kept relatively small, e.g. only approximately 40% that of the BudCap™. Thus, material cost is reduced by the present invention by approximately 60% while its effectiveness as a deterrent is reduced by only approximately 20%. (Actually, even less, in practice, since the BudCap™ device has been offered to date with only approximately 20% of its outer surface area scented and only a portion of that area being fully exposed due to its conic configuration. This compares with approximately 62.5% of the outer surface of invented device 10 being scented and with the entire scented surface area being fully exposed, due to the splayed-end looping configuration of the invented device.) Printed matter region 12aa in accordance with one embodiment of the invention is an approximately 0.75″ by 3″ rectangle near the middle of substrate 12 on surface 12a thereof. This ensures good visibility of a company logo or the like when device 10 is deployed around a seedling stem. Printed matter region 12aa in accordance with one embodiment of the invention is separated from scented region 12c. This is so that the two processes, printing the printed matter and applying the scent, remain compatible and so that the printed matter is not obscured by the scent. (It is contemplated as being within the spirit and scope of the invention to place printed matter region 12aa anywhere or nowhere, and to place adhesive region 16 in an alternative location along other surface 12b of substrate 12, e.g. either end thereof, so that protective device 10 exhibits a single, long, scented end and grasps seedling stem S near an opposite end thereof, while still maximizing scented region surface area and deterrence against animal browsing.)

Those of skill in the art will appreciate that the scent can be a mixture of pig blood, a curing agent for drying the pig's blood and a spray fixative or anti-stick coating over the pig's blood and curing agent that renders the scented region 12c of device 10 safe and convenient to handle. But the repellent scent can take any suitable natural or synthetic fragrance alone or in combination with other agents that render it effective upon application to repelling animal browsers, e.g. a deer or other browsing animal. Thus, it is contemplated to be within the scope and spirit of the invention to apply in any form any scent capable of repelling any and all animal browsers.

Alternative embodiments of invented device 10 are contemplated as being within the spirit and scope of the invention. For example, substrate 12 can have the scent impregnated therein and thus distributed therethrough, as by known micro-encapsulation and time release techniques, rather than being applied to one or more surfaces thereof. Another exemplary alternative embodiment would involve applying the scent to fewer than both surfaces 12a and 12b and to fewer than four regions 12ca, 12cb, 12cc and 12cd. This is because the scent is yet somewhat effective even if not substantially exposed on an outer surface of device 10 when the device is deployed around the stem of a seedling. Nevertheless, maximizing the surface area exposure of the repellent scent to the animal browser ensures maximum deterrence to browsing. The splayed configuration of device 10 in accordance with one embodiment described and illustrated herein thus doubles and quadruples the exposed surface are of repellent scent and thus increases both the potency of the deterrent and its useful life.

The scent can be applied to the surfaces 12a and 12b, e.g. the outer and inner, exposed surfaces of substrate 12, without shelf-life loss of potency and without environmental or safety hazard. This can be accomplished by formulating and applying the scent in such manner as to render it environment-activated, as one or more of sunlight and rainwater. Such an EPA-approved scent formulation and application is available from Precision Press, Inc. of Mankato, Minn. The useful life of the scent is substantially undiminished while device 10 so equipped is undeployed as part of a roll of other inventory because the scent remains inactive until it is activated by the environment around the time of deployment of device 10 around a seedling in the activating environment. (Those of skill in the art will appreciate that filtered sunlight and/or artificial grow lights and water affecting the seedling in the relative confines of a nursery substitutes for natural sunlight and rainwater so that the scent is activated therein also.) Thus, the useful life of invented device 10 is extended even if the scent is applied to a surface such as surface 12b of the substrate, which is exposed to the environment, so long as reasonable precautions are taken.

If the scent is applied instead to only one surface 12b, e.g. only in scent regions 12cc, 12cd, then it can be of a different formulation, if desired, and can be activated, for example, by removing cover 14 at the time of deployment around the seedling. Indeed, scent can be applied to all four scent regions 12ca, 12cb, 12cc and 12cd but using different formulations to capitalize on different release mechanisms. For example, a time-release scent can be applied to uncovered scent regions 12ca and/or 12cb that effectively activate and slowly release from the time of application of the scent or from the time of environmental exposure of the undeployed device, while a non-time-release scent can be applied to covered scent regions 12cc and/or 12cc that effectively activate and quickly release from the time of deployment, when laminar cover 16 is removed from substrate 12.

In this way, two or more scent repellent mechanisms can be at work concurrently to provide maximum deterrent effect at the time of maximum vulnerability (when a seedling is planted in a forest) and also to provide a controlled deterrent effect then and for some time thereafter. Or the two or more applications can both be of a time-release scent but characterized by different time-release dynamics, one relatively fast-acting and the other relatively slow-acting, thereby providing a complementary timed release of scent as deterrent.

Accordingly, any combination of scent formulations and applications in one or more of scent regions 12ca, 12cb, 12cc and/or 12cd are contemplated as being within the spirit and scope of the invention.

FIG. 2 shows device 10 deployed around a seedling stem S near an extremity L thereof also known in the art as a leader (and just below a last lateral branching that secures device 10 from blowing or being pecked or nudged or otherwise falling off). FIG. 2 also shows a BudCap™ protective device BC previously deployed around the seedling stem S at an earlier stage of the seedling's maturity, e.g. at a time of its first flushing (typically in the spring of the growing season). It will be understood by those skilled in the art that BudCap™ device BC typically is attached at least approximately six months before device 10, and possibly within the same seedling planting season. Thus, its scent might be diminished along with its structural integrity. In most cases, it will be less effective than when it was first applied to the seedling because the seedling has grown typically between four and eighteen inches. This growth depends upon how much leader L growth occurred within the first six months and whether there was an additional six inch ‘reflush’ in the fall following the spring planting.

As may be seen from FIG. 2, device 10, when made in accordance with one embodiment of the invention and when deployed on a seedling stem S as described and illustrated herein, provides a high level of effective scented repellent effect. It does so because of the uniquely splayed, radially extending, opposing ends, which freely expose all four of scented regions 12ca, 12cb, 12cc and 12cd to the environment while grasping the seedling stem in a central adhesive region of the substrate to affix device 10 to the seedling at an elevation thereon near the flush or reflush bud where it is believed to be most effective. It is this splayed configuration of the free opposing ends of device 10 having exposed scent thereon that keeps unit cost down and yet keeps scent level and thus animal browsing deterrent effect high.

The periodic use of plural devices 10 thus augments and extends the time for effective deterrence to browsing—e.g. six months, twelve months, eighteen months, twenty-four months—after initial use of a BudCap™ device on the new seedling. The periodic use of serial ones of plural devices 10 in combination alone provides a continuous-protection system for seedlings, through their first two years of growth, after which their vulnerability to browsing greatly diminishes. Alternatively, the use of one or more BudCap™ devices and one or more invented devices 10 in combination similarly provides a continuous-protection system for seedlings through their first two years of growth. Thus, seedlings can now be protected from the earliest stage of growth through the latest stage of growth when protection is needed by the use of plural invented devices 10 alone or by the use of one or more invented devices 10 in combination with one or more BudCap™ devices BC.

Those of skill will appreciate that seedling protection is labor intensive. Typically, a forester or laborer walks through a seedling row (e.g. in a nursery) or through a seedling area (e.g. in a forest) affixing the terminal stem of each seedling with a protective device. This is why, in accordance with one embodiment of the invention, plural instances of device 10 are formed in the form of a roll providing for their quick and easy serial dispensing therefrom. To accomplish this dispensing, the plural ones of device 10 are oriented end-to-end and adjacent pairs of devices 10 are detachably, e.g. break-ably (tear-ably), connected to one another in their adjacent end regions by a frangible web of connecting material. In accordance with one embodiment of the invention, this web of connecting material is provided by perforating an otherwise continuous strip of end-to-end devices along parallel lines properly and regularly spaced apart the lengthwise extent of the individual strips along the roll.

FIG. 3 shows plural ones of device 10 in a contiguous roll 18, with webs 20 of perforation-weakened substrate/cover material connecting adjacent pairs of devices 10. Forming plural devices 10 into a roll renders them easily, serially dispensable to reduce the time and cost of the labor involved in applying them to plural seedlings in a nursery or in the field (e.g. a forest). Those of skill will appreciate that adjacent pairs of end-to-end configured are connected to one another by webs 20 that can be made of the same material as the devices 10 themselves. It will also be appreciated that the roll of contiguous plural devices 10 can be formed by any suitable manner, as by a process compatible with the devices' material makeup. For example, paper slurry can be molded in a contiguous strip and then die-stamped to form the perforations as the contiguous strip is rolled. Any suitable method of forming the contiguous strips and rolls of dispensable devices 10 is contemplated, and is within the spirit and scope of the invention.

Roll 18 of dispensable devices 10 can be easily tethered to a user (e.g. a forester or other laborer) by a necklace, e.g. a rope R or lanyard or the like, dimensioned and textured comfortably to extend around the user's neck, as suggested by FIG. 3. (A core around which roll 18 of plural dispensable devices 10 can be spooled is not shown in FIG. 3, but such might optionally be used in connection with such spooling, or such dispensing, or both.) It will be appreciated that the plural connected devices 10 in roll 18 have, in accordance with one embodiment of the invention, (top) surface 12a of substrate 12 exposed on the outer circumference of the roll, with laminar cover 14 tucked in the interior of the roll. This renders dispensing simple and quick, thus further saving labor time and cost. Thus, dispensing and applying serial devices 10 is as quick and easy as tearing the device from the exposed end of the roll, removing its laminar cover and pinching it around a seedling's terminal stem to adhere the opposing ends of the device to one another substantially along their length. This secures the protective device and also adheres the device around its bend to the stem at a desired height on the seedling beneath the terminal branches thereof, as shown in FIG. 2. The company logo is highly visible and the scent is ready to be released to discourage animal browsing.

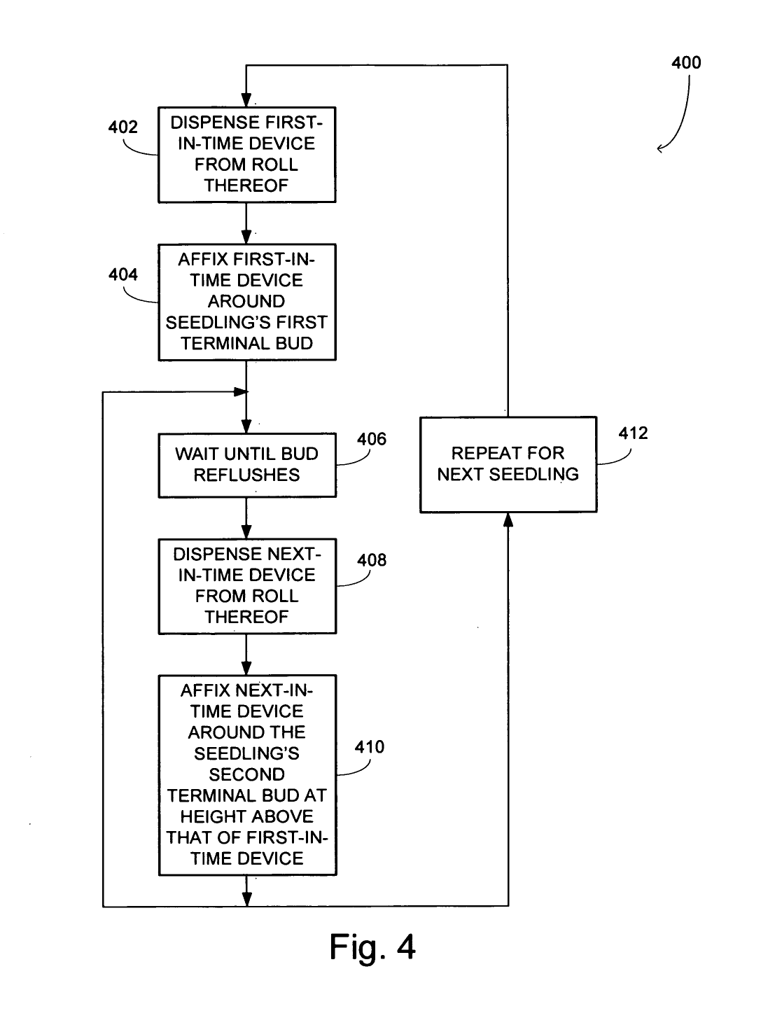

FIG. 4 is a simplified flowchart illustrating the method in accordance with one embodiment of the invention. The method includes a) at 402, dispensing a first-in-time protective device (e.g. a BudCap™ device) from a roll thereof; b) at 404, affixing the first-in-time protective device around a seedling's first terminal bud; c) at 406, waiting approximately six months or until the bud reflushes; d) at 408, dispensing a second-in-time protective device (e.g. invented device 10) from a roll thereof; and d) at 410, affixing the next-in-time protective device around a seedling terminal bud at a height above that of the first-in-time device (as illustrated in FIG. 2). The next-in-time dispensing and affixing steps can optionally be repeated indefinitely or for a desired time after another wait, as indicated. Another seedling can be protected by e) at 412, repeating the steps.

Those of skill in the art will appreciate that steps 402 and 404 are optional, in accordance with one embodiment of the invention, and that step 406 is optional at the start of the growing season, if it is desired to utilize only the invented protective device 10 to protect seedlings from animal browsing and not to use the recently introduced bud capping protective device such as the BudCap™ protective device. Accordingly, the invented method features protection of one or more seedlings using one or more differently configured protective devices, as well as protection of a single seedling over its vulnerable life using one or more protective devices of a single configuration.

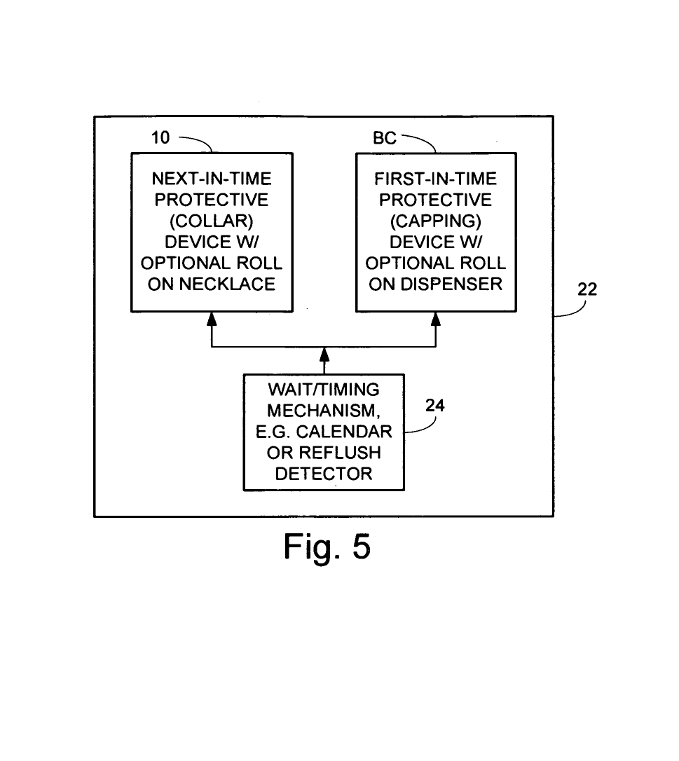

FIG. 5 is a simplified block diagram of system 22 in accordance with one embodiment of the invention. System 22 can be seen to include optional patented (capping) protective device BC (optionally in a roll on a dispenser, in accordance with the teachings of the above-referenced patent), invented (collar) protective device 10 (optionally in a roll on a necklace, e.g. a rope or lanyard or the like) and a wait/timing mechanism 24, e.g. a calendar indicating an approximately six-month interval between successive affixings of one or more of such protective devices and/or a reflush detector, e.g. manual or assisted observation indicating that it is time once again to affix a fresh one of the protective devices to a given seedling to renew its resistance to animal browsing. Those of skill in the art will appreciate that timing is helpful in maintaining physical or scent-repellent discouragement to animal browsing, since a seedling is vulnerable over approximately two years of its life to such harmful and potentially lethal browsing.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

From the foregoing, those of skill in the art will appreciate that several advantages of the present invention include the following.

The present invention provides a relatively inexpensive and easy to use device, system and method of protecting a seedling throughout its vulnerable growth period form animal browsers. The invented device is easy and quick to apply and can be easily dispensed form a roll strung around the user's neck. The invented device can be used alone or in combination with the patented BudCap™ device and method of its application. The invention inexpensively extends protection of vulnerable seedling from start to the end of its growth stage when it is no longer vulnerable to browsing. It does so by including a repellent scent to discourage animal browsing, a scent that is not activated until the device is deployed around a terminal stem of a seedling. It provides a useful alternative to sprays and other application techniques that require more difficult, repetitive hand gripping motions and are less targeted and thus less effective. It does so by providing also a lightweight and inexpensive paper carrier that already has the scent applied to it but that does not release its scent until its time.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

I claim:

1. A seedling protection device comprising:
a flexible strip of thin flat material having opposing surfaces and opposing ends,
the strip being flexible to define an interior one of the opposing surfaces and to produce an intermediate overlapping interior region thereof,
the strip including an adhesive at least in the intermediate overlapping interior region,
the strip having a laminar cover extending substantially over the interior one of the opposing surfaces to protect the adhesive, pre-deployment, the laminar cover being removable from the interior one of the opposing surfaces in a deployment phase of use,
the strip including on at least one surface region adjacent one or more opposing ends thereof a scented compound,
the strip with the laminar cover removed and when flexed, during deployment, forming a collar configured to extend around and affix to a seedling stem near an extremity thereof.

2. The device of claim 1, wherein the strip is elongate and wherein the opposing ends are aligned with a long axis of the elongate strip.

3. The device of claim 2, wherein the adhesive extends only partway along the elongate strip, and wherein at least one opposing end of the strip including the scented compound includes no adhesive.

4. The device of claim 3, wherein the adhesive extends along only a middle part of the elongate strip, and wherein both opposing ends of the strip include the scented compound and include no adhesive on both exposed surfaces thereof.

5. The device of claim 4, wherein both opposing ends of the strip include the scented compound on four exposed surfaces thereof.

6. The device of claim 5, wherein the scented compound includes an animal repellent.

7. The device of claim 6, wherein the material of the flexible strip is water-repellent paper stock.

8. The device of claim 7, wherein an exterior one of the opposing surfaces includes printed matter thereon.

9. The device of claim 8, wherein the printed matter is in a middle part of the elongate strip opposite the adhesive.

10. The device of claim 8, wherein the printed matter includes supplier indicia including at least one of a company name, a trademark and a logo.

11. The device of claim 1, wherein plural ones of the strips are joined together at adjacent ones of their opposite ends in a contiguous roll with one or more perforations therebetween facilitating serial dispensing one at a time thereof from the roll.

12. A seedling protection system for protecting a seedling against animal browsing from an early stage to a later stage of growth, the system comprising:
a first device for affixment around a stem of the seedling, the first device including a repellent scent affixed on or in at least one surface thereof to discourage animal browsing, the first device including the repellent scent being configured to maximize surface area exposure of the repellent scent to a browsing animal.

13. The system of claim 12 which further comprises:
a second device for capping an upper extremity of a seedling while permitting ambient light to reach the seedling, at an earlier stage of growth thereof than a stage of growth during which the first device is affixed, the second device being physically configured to discourage animal browsing.

14. The system of claim 12, wherein the first device includes a first generally planar laminar structure including a first substrate and a first laminate, the first laminate including at least a removable portion removal of which, during deployment of the first device, exposes a first adhesive surface of the first substrate, the first adhesive surface being configured when the first lamninar structure is manually formed into a collar to adhere opposing regions of the first laminar structure in the vicinity of a terminal seedling stem, the collar extending around and being affixed by the first adhesive region to the terminal seedling bud.

15. The system of claim 14, wherein the second device includes a second generally planar laminar structure including a second substrate and a second laminate, the second laminate including at least a removable portion removal of which, during deployment of the second device, exposes a second adhesive surface of the substrate, the second adhesive surface being configured when the second laminar structure is manually formed into a cone to adhere opposite edges of the second laminar structure, the cone extending upwardly and outwardly from a stem of the extremity to at least partly enclose the extremity.

16. The system of claim 15, wherein the second device includes a repellent scent further to discourage animal browsing.

17. The system of claim 12 which further comprises:
plural instances of the first device in the form of a roll with adjacent ones of the plural first instances joined with a frangible web of material for serial dispensing of the plural first instances from the first roll.

18. The system of claim 17 which further comprises:
plural instances of the second device in the form of a second roll with adjacent ones of the plural second instances joined with a second frangible web of material for serial dispensing of the plural second instances from the second roll.

19. The system of claim 18 which further comprises:
a dispenser including a spindle mounted to a disk for holding the second roll of plural second instances and for dispensing the same one at a time.

20. A method of protecting a seedling, the method comprising:
affixing a first-in-time seedling protective device around a first terminal bud of a seedling by forming a flat laminar structure into an inverted cone and adhering its overlapping edges together; and, at a later time
affixing a next-in-time seedling protective device around a second terminal bud of the same seedling by bending a flat laminar elongate structure containing a repellent scent therearound and affixing it to the second terminal bud,
the next-in-time seedling protective device being affixed to the seedling above the height of the first-in-time seedling protective device.

21. The method of claim 20 whereby plural seedlings are so protected, wherein the affixing of the first-in-time seedling protective device and the affixing of the next-in-time seedling protective device is repeated for each of plural seedlings, and wherein, immediately before each affixing, the method further comprises:
dispensing one or more first-in-time seedling protective devices from a contiguous roll thereof by tearing a frangible web therebetween; and
dispensing one or more next-in-time seedling protective devices from a contiguous roll thereof by tearing a frangible web therebetween.

22. The method of claim 20, wherein, for a given seedling, between the affixing of the first-in-time seedling protective device and the affixing of the next-in-time seedling protective device, the method further comprises:
waiting between approximately six months and one year or until the seedling's bud reflushes.

23. The method of claim 20, wherein, for a given seedling, the affixing of the next-in-time seedling protective device is repeated using two or more next-in-time seedling protective devices.

24. The method of claim 23, wherein repeated affixings of the two or more next-in-time seedling protective devices are spaced in time approximately six months or are timed approximately to correspond with one or more reflushes of the seedling bud.
